(19) United States
(12) Patent Application Publication
(10) Pub. No.: US 2006/0008438 A1
(43) Pub. Date: Jan. 12, 2006

(54) Multi-phased personal care composition

(76) Inventors: Andres Ernesto Velarde, Cincinnati, OH (US); Steven Kirk Hedges, Fairfield, OH (US); Edward Dewey Smith III, Mason, OH (US); Karl Shiqing Wei, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/178,047

(22) Filed: Jul. 8, 2005

Related U.S. Application Data
(60) Provisional application No. 60/586,740, filed on Jul. 9, 2004.

(51) Int. Cl. A61K 8/89; A61K 8/46; A61K 36/899

(52) U.S. Cl. 424/70.12; 424/70.22; 424/74

(57) ABSTRACT

The present invention is a multi-phased personal care composition that contains at least two visually distinct phases. At least one of the visually distinct phases is a benefit phase that has a Flow Onset Temperature of at least about 97° F.; and wherein said visually distinct phases form a pattern; and wherein said visually distinct phases are packaged in physical contact with one another and are stable.

### CROSS REFERENCE TO RELATED APPLICATION(S)

This application claims the benefit of U.S. Provisional Application Ser. No. 60/586,740, filed Jul. 9, 2004.

### FIELD OF THE INVENTION

The present invention relates to a multi-phased personal care composition comprising at least two visually distinct phases. At least one of the visually distinct phases is a benefit phase that has a Flow Onset Temperature of at least about 97° F.; and wherein said visually distinct phases form a pattern; and wherein said visually distinct phases are packaged in physical contact with one another and are stable.

### BACKGROUND OF THE INVENTION

Personal care compositions are becoming more popular in the United States and around the world. Desirable personal care composition must meet a number of criteria. For example, in order to be acceptable to consumers, a personal care composition must exhibit good cleaning properties, must exhibit good lathering characteristics, must be mild to the skin (not cause drying or irritation) and preferably should even provide a conditioning benefit to the skin. Personal care compositions have also been used to alter the color and appearance of skin.

Personal care compositions that attempt to provide skin-conditioning benefits are known. Many of these compositions are aqueous systems comprising an emulsified conditioning oil or other similar materials in combination with a lathering surfactant. In order to overcome emulsification of the skin conditioning agents by the cleansing surfactant, large amounts of the skin conditioning agent are added to the compositions.

One attempt at providing conditioning and cleansing benefits from a personal care composition while maintaining stability has been the use of dual-chamber packaging. These packages comprise separate cleansing compositions and conditioning compositions, and allow for the co-dispensing of the two in a single or dual stream. The separate conditioning and cleansing compositions thus remain physically separate and stable during prolonged storage and just prior to application, but then mix during or after dispensing to provide conditioning and cleansing benefits from a physically stable system. Although such dual-chamber delivery systems provide improved conditioning benefits over the use of conventional systems, it is often difficult to achieve consistent and uniform performance because of the uneven dispensing ratio between the cleansing phase and the conditioning phase from these dual-chamber packages. Additionally, these packaging systems add considerable cost to the finished product.

When a personal care composition comprises a visually distinct phase, it is desirable for the visually distinct phase to be stable, i.e., not change in appearance over time. Personal care compositions are often transported in conditions exposing the compositions to conditions of thermal extremes and/or considerable vibration, dropping, and other mechanical stress, through which it may be desirable for the personal care composition properties to confer stability to the phase. Thus, a balanced composition comprising the personal care composition is necessary, such that these tradeoffs can be managed, in order to manufacture a composition comprising a benefit phase which has both preferred skin feel and adequate stability during transportation.

Accordingly, the need still remains for multi-phased personal care composition that comprises at least two phases in physical contact, visually distinct and form a pattern. These multi-phased personal care compositions should remain stable during the stresses of transportation of the composition but also maintain preferred skin feel.

It is therefore an object of the present invention to provide a multi-phased personal care composition comprising at least two visually distinct phases that are in physical contact with each other and form a pattern. Additionally at least one of the visually distinct phases comprises a benefit phase having a Flow Onset Temperature of at least about 97° F. and a Consistency Value from about 30 to about 350 Pa-s. Multi-phased personal care composition comprising benefit phases within these parameters produces a composition that maintains the visually distinct phases and the pattern they form while at the same time the composition is stable during stress conditions such as transportation, extreme heat and storage and still deliver preferred skin feel.

### SUMMARY OF THE INVENTION

The present invention relates to a multi-phase personal care composition comprising: at least two visually distinct phases; wherein at least one visually distinct phase comprises a benefit phase having a Flow Onset Temperature of at least about 97° F.; wherein said benefit phase has a Consistency Value (K) from about 30 to about 350 Pa-s and wherein said visually distinct phases form a pattern; and wherein said visually distinct phases are packaged in physical contact with one another and are stable.

The present invention further relates to a multi-phase personal care composition comprising: at least two visually distinct phases; wherein at least one visually distinct phase comprises a benefit phase having a Structural Rigidity of greater than about 60 Pa; wherein said benefit phase has a Consistency Value (K) from about 30 to about 350 Pa-s; and wherein said phases form a pattern; and wherein said phases are packaged in physical contact with one another and are stable.

The present invention further relates to a multi-phase personal care composition comprising: at least two visually distinct phases comprising a benefit phase; a cleansing phase comprising; i) a surfactant component comprising; a) at least one nonionic surfactant having an HLB from about 3.4 to about 15.0; b) at least one anionic surfactant; c) at least one amphoteric surfactant; ii) an electrolyte; and wherein said benefit phase has a Flow Onset Temperature of at least about 97° F.; and wherein said benefit phase has a Consistency Value (K) from about 30 to about 350 Pa-s; and wherein said phases form a pattern; and wherein said phases are packaged in physical contact with one another and are stable.

The present invention is also directed to a method of cleansing, moisturizing and delivering skin benefit agents and particles to the skin by applying to the skin a composition as described above.

The present invention is also directed to an article of commerce comprising (a) a container having an s-wave orifice; (b) a multi-phase personal care composition contained in said container having an s-wave orifice; and (c) a set of instructions in association with said container comprising instructions to cleanse, to condition, to moisturize the skin and rinse; and wherein said container allows for easy dispensing of said composition.

### DETAILED DESCRIPTION OF THE INVENTION

The present invention relates to a multi-phase personal care composition comprising: at least two visually distinct phases; wherein at least one visually distinct phase comprises a benefit phase having a Flow Onset Temperature of at least about 97° F.; and wherein said benefit phase has a Consistency Value (K) from about 30 to about 350 Pa-s; and wherein said visually distinct phases form a pattern; and wherein said visually distinct phases are packaged in physical contact with one another and are stable.

These and other essential limitations of the compositions and methods of the present invention, as well as many of the optional ingredients suitable for use herein, are described in detail hereinafter.

The term “anhydrous” as used herein, unless otherwise specified, refers to those compositions or components containing less than about 10%, more preferably less than about 5%, even more preferably less than about 3%, even more preferably zero percent, by weight of water.

The term “ambient conditions” as used herein, refers to surrounding conditions at one (1) atmosphere of pressure, 50% relative humidity, and 25° C.

The term “cosmetically efficacious level” as used herein, is a level conferring a benefit during use of the composition.

The term “domain”, as used herein means a volume of material, component, composition or phase comprising a molecular mixture which can be concentrated but not further separated by physical forces such as ultracentrifugation. For example, surfactant lamellar, surfactant micellar, surfactant crystal, oil, wax, water-glycerine mixture, hydrated hydrophilic polymer all constitute domains which can be concentrated and observed by ultracentrifugation, but which cannot be further separated into distinct molecular components by the same forces.

The term “hydrophobically modified interference pigment” or “HMIP”, as used herein, means a portion of the interference pigment surface has been coated, including both physical and chemical bonding of molecules, with a hydrophobic material.

The term “interference pigment”, as used herein, means a pigment with pearl gloss prepared by coating the surface of a particle substrate material (generally platelet in shape) with a thin film. The thin film is a transparent or semitransparent material having a high refractive index. The higher refractive index material shows a pearl gloss resulting from mutual interfering action between reflection and incident light from the platelet substrate/coating layer interface and reflection of incident light from the surface of the coating layer.

By the term “multi-phased” or “multi-phase” as used herein, is meant that the at least two phases herein occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof.

In a preferred embodiment the striped pattern may be relatively uniform and even across the dimension of the package. Alternatively, the striped pattern may be uneven, i.e. wavy, or may be non-uniform in dimension. The striped pattern does not need to necessarily extend across the entire dimension of the package. The size of the stripes can be at least about 0.1 mm in width and 10 mm in length, preferably at least about 1 mm in width and at least 20 mm in length. The phases may be various different colors, or include particles, glitter or pearlescence.

The term “multi-phased personal care composition” as used herein, refers to compositions intended for topical application to the skin or hair.

The term “phases” as used herein, refers to a region of a composition having one average composition, as distinct from another region having a different average composition, wherein the regions are visible to the naked eye. This would not preclude the distinct regions from comprising two similar phases where one phase could comprise pigments, dyes, particles, and various optional ingredients, hence a region of a different average composition.

The term “stable” as used herein, unless otherwise specified, refers to compositions that maintain at least two “separate” phases when sitting in physical contact at ambient conditions for a period of at least about 180 days wherein the distribution of the two phases in different locations in the package does not change over time. By “separate” is meant that the well-distributed nature of the visually distinct phases and also the pattered appearance is compromised, such that larger regions of at least one phase collect until the balanced dispensed ratio of the two or more compositions relative to each other is compromised.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The term “s-wave orifice” as used herein refers to a dispensing orifice integral to a packaging piece such as a cap through which the composition is expelled by application of pressure, wherein the orifice has a curvature in the shape of a wave or waves or portion of a wave.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The multi-phased personal care composition and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

### Product Form

The multi-phased personal care composition of the present invention is typically in the form of a liquid. The term “liquid” as used herein means that the composition is generally flowable to some degree when sufficient force is applied. “Liquids”, therefore, can include liquid, semi-liquid, cream, lotion or gel compositions intended for topical application to skin. The compositions typically exhibit a viscosity of from about 1,500 cps to about 1,000,000 cps, as measured by the Viscosity Method as described in copending application Ser. No. 60/542,710 filed on Feb. 6, 2004. These compositions contain at least two phases, which are described in greater detail hereinafter.

When evaluating a multi-phased personal care composition, by the methods described herein, preferably each individual phase is evaluated prior to combining, unless otherwise indicated in the individual methodology. However, if the phases are combined, each phase can be separated by physical means such as centrifugation, ultracentrifugation, pipetting, filtering, washing dilution, or combination thereof, and then the separate components or phases can be evaluated. Preferably, the separation means is chosen so that the resulting separated components being evaluated is not destroyed, but is representative of the component as it exists in the multi-phased personal care composition. The product forms contemplated for purposes of defining the compositions and methods of the present invention are preferably rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition. However, it is contemplated that the subject compositions may be used as leave-on personal care compositions as well without deviating from the spirit of the invention.

In a preferred embodiment of the present invention the multi-phased personal care composition, has at least two visually distinct phases wherein at least one phase is visually distinct from a second phase. The visually distinct phases are packaged in physical contact with one another and are stable.

### Phases

The multi-phase personal care compositions of the present invention comprises at least two phases, wherein in the composition can have a first phase a second phase and so on. The ratio of a first phase to a second phase is about 97:3 to about 3:97, preferably about 80:20 to about 20:80, more preferably about 70:30 to about 30:70, even more preferably about 60:40 to about 40:60, still more preferably about 50:50. Each phase could be one or more of the following nonlimiting examples including: a cleansing phase, a benefit phase, and a non-lathering structured aqueous phase, which are described in greater detail hereinafter.

Benefit Phase

The multi-phase personal care compositions of the present invention can comprise a benefit phase. When the benefit phase is not an emulsion, the benefit phase in the present invention is preferably anhydrous. The benefit phase comprises hydrophobic compositions comprising hydrophobic components. The hydrophobic compositions suitable for use in the present invention have a Vaughan Solubility Parameter of from about 5 to about 15 preferably from about 5 to about 10, more preferably from about 6 to about 9. These solubility parameters are well known in the formulation arts, and are defined by Vaughan in Cosmetics and Toiletries, Vol. 103, p 47-69, October 1988. Non-limiting examples of hydrophobic components having VSP values ranging from about 5 to about 15 include the following:

### Vaughan Solubility Parameters*

| | |
|---|---|
| Cyclomethicone | 5.92 |
| Squalene | 6.03 |
| Petrolatum | 7.33 |
| Isopropyl Palmitate | 7.78 |
| Isopropyl Myristate | 8.02 |
| Castor Oil | 8.90 |
| Cholesterol | 9.55 |

As reported in Solubility, Effects in Product, Package, Penetration and Preservation, C. D. Vaughan, Cosmetics and Toiletries, Vol. 103, October 1988.

The benefit phase comprises from about 5% to about 100%, preferably at least about 25%, most preferably at least about 50%, by weight of the benefit phase, of a hydrophobic composition. The hydrophobic composition comprises from about 5% to about 100%, preferably at least about 25%, most preferably at least about 50%, by weight of the hydrophobic composition, of a hydrophobic component.

The benefit phase is preferably selected among those having defined stability properties. These preferred stability properties are especially useful in providing the multi-phased personal care composition compositions with improved stability. Rheology measurements described herein are useful for describing benefit phases which comprise structurants that change their behavior as a function of temperature, such as waxes, fatty alcohols, etc. The Flow Onset Temperature determines at what temperature the benefit phase becomes fluid-like, hence commencing potential instability of the pattern. This is important for example to determine at what maximum temperature a patterned visually distinct composition comprising the benefit phase comprising hydrophobic composition comprising hydrophobic components may be stored and/or transported, for example during distribution including warehousing during the summertime or in warm climates. It is well established by published research that cargo truck interior air temperatures in the south United States, for example, can be higher than 110° F. for more than 8 hours during a day (ISTA Temperature Project Data Summary, International Safe Transit Association, East Lansing, Mich., USA, 2001). The benefit phase of the present invention has a Flow Onset Temperature of at least about 97° F., preferably at least about 99° F., more preferably at least about 100° F., even more preferably at least about 101° F., still even more preferably at least about 102° F., even still even more preferably at least about 104.5° F., and even still even more preferably 108° F., as measured by the Flow Onset Temperature Method described hereafter.

Structural Rigidity is used to determine how rigid the benefit phase is at 100° F., a temperature expected to be reached during dry goods transportation. Benefit phases having sufficient rigidity can be expected to retain their structure, hence pattern, through the increased stresses that result from transportation of the composition, for example during shipping or moving cases about a warehouse or storeroom and storage. Compositions and phases having sufficient structural rigidity are said to have good thermal stability. Conventionally, rigidity of structure for a material can be determined in accordance with Hooke's law (Materials Science for Engineers, L. Van Vlack, Addison Wesley Publishing, USA, 1970, p. 188) which expresses the ratio of stress to strain in reversible deformation as a constant ratio of stress to strain, sometimes called Young's Modulus or the Elastic Modulus. In the same way, a stress can be applied rotationally to the benefit phase using a rheometer, and the relationship between stress and strain at a low value of stress, 1 Pa, expressed as the stress divided by the resulting amount of strain the hydrophobic composition experiences. The Structural Rigidity is defined as 1 Pa divided by the amount of unitless strain demonstrated at 1 Pa of stress in this test, at 100° F. The benefit phase that is stable during transportation at elevated temperature preferably has a Structural Rigidity of greater than about 60 Pa, preferably greater than about 65 Pa, more preferably greater than about 75 Pa, even more preferably greater than about 100 Pa, still more preferably greater than about 125 P as determined by the Structural Rigidity Method described hereafter.

Skin Feel Rheology is used to determine the preferred rheology profile of the benefit phase so that when the multi-phased personal care composition is deposited on the skin, the skin feels moisturized but not heavy or sticky or draggy. The consistency value is a measure of the skin feel of the benefit phase as defined by Consistency Value (K) and Shear Index (n). The benefit phase has a Consistency Value (K) from about 30 to about 350 Pa-s, preferably from about 35 to about 300 Pa-s, more preferably from about 40 to about 250 Pa-s, still more preferably from about 45 to about 150 Pa-s and even still more preferably from about 15 to about 125 Pa-s. The benefit phase has a Shear Index from about 0.025 to about 0.93, preferably from about 0.05 to about 0.70 and more preferably from about 0.09 to about 0.60. The values are determined at 25° C.

The benefit phase can be characterized by Consistency Value (K) and Shear Index (n) values as defined by the above-described ranges, wherein these defined ranges are selected to provide reduced stickiness during and after application of the multi-phase personal care composition on hair or skin.

The Shear Index (n) and Consistency Value (K) are known and accepted means for reporting the viscosity profile of materials having a viscosity that varies with applied shear rate using a Power Law model.

The viscosity (μ) for a benefit phase can be measured by applying a shear stress and measuring the shear rate using a rheometer, such as a TA Instruments AR2000 (TA Instruments, New Castle, Del., USA 19720). Viscosity is determined at different shear rates in the following manner. First, the benefit phase is obtained. If there exists more than one distinct (immiscible, e.g.) benefit phase in the composition, such as for example a silicone oil phase and a hydrocarbon phase, they are prepared and evaluated separately from each other.

For measurement, a 40 mm diameter parallel plate geometry with a gap of 1 mm is used unless there are particles greater than 0.25 mm, in which case a gap of 2 mm is used. The rheometer uses standard parallel plate conventions to report shear rate at the edge as shear rate of the test; and converts torque to stress using the factor 2/(πR³). Using a spatula, a sample comprising a small excess of the benefit phase is loaded onto the rheometer base plate which is at 25° C., the gap is obtained, and excess composition outside the top measurement geometry is removed, locking the top plate in position during the removal of excess sample. The sample is equilibrated to the base plate temperature for 2 minutes. A preshear step is performed comprising 15 seconds of shear at a shear rate of 50 inverse seconds (1/sec). As is known to one skilled in the art, the shear rate with a parallel plate geometry is expressed as the shear rate at the edge, which is also the maximum shear rate. After the preshear step, the measurement is performed, which comprises ramping the stress from 10 Pa to 1,000 Pa over a 2.0 minute interval at 25° C., while collecting 60 viscosity data points, in an evenly spaced linear progression. A shear rate of at least 500 l/seconds is obtained in the test, or the test is repeated with a fresh sample of the same component with a higher final stress value, maintaining the same rate of stress increase per time, until a shear rate of at least 500 l/sec is obtained during the measurement period. During the measurement, observe the sample to make certain the area under the top parallel plate is not evacuated of sample at any edge location during the measurement, or the measurement is repeated until a sample remains for the duration of the test. If after several trials a result cannot be obtained due to sample evacuation at the edge, the measurement is repeated leaving an excess reservoir of material at the edge (not scraping). If evacuation still cannot be avoided, a concentric cylinder geometry is used with a large excess of sample to avoid air pockets during loading. The results are fitted to the power law model by selecting only the data points between 25-500 l/sec shear rate, viscosity in Pa-s, shear rate in 1/sec, and using a least squares regression of the logarithm of viscosity vs. the logarithm of shear rate to obtain values of K and n according to the Power Law equation:
μ=K(γ′)⁽ⁿ⁻¹⁾

The value obtained for the log-log slope is (n−1) where n is the Shear Index and the value obtained for K is the Consistency Value, expressed in units of in Pa-s.

Nonlimiting examples of hydrophobic component suitable for use within the benefit phase herein can include a variety of natural and synthetic lipids, hydrocarbons, fats, oils, hydrophobic plant extracts, fatty acids, essential oils, silicone oils, triglycerides, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, oxidized waxes, and combinations thereof.

Non-limiting examples of silicone oils suitable for use as hydrophobic components herein include dimethicone copolyol, dimethylpolysiloxane, diethylpolysiloxane, silicone gums, silicone resins such as MQ resin, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use as hydrophobic components herein include castor oil, soy bean oil, derivatized oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as hydrophobic components herein include acetylated monoglycerides.

Non-limiting examples of alkyl esters suitable for use as hydrophobic components herein include isopropyl esters of fatty acids and long chain esters of long chain (i.e. C₁₀-C₂₄) fatty acids, e.g. cetyl ricinoleate, non-limiting examples of which include isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic components herein include oleyl myristate, oleyl stearate, oleyl oleate, and combinations thereof.

Non-limiting examples of polyglycerin fatty acid esters suitable for use as hydrophobic components herein include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic components herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable hydrophobic components suitable for use within the benefit phase herein include milk triglycerides (e.g., hydroxylated milk glyceride) and polyol fatty acid polyesters.

Still other suitable hydrophobic components suitable for use within the benefit phase herein include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

Preferably the hydrophobic component suitable for use within the benefit phase is selected from the group consisting of petrolatum, mineral oil, micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

Cleansing Phase

The multi-phase personal care composition of the present invention can comprise a cleansing phase. The cleansing phase comprises a surfactant component comprising a surfactant or a mixture of surfactants.

Surfactant Component

The surfactant component comprises a surfactant or a mixture of surfactants. The surfactant component comprises surfactants suitable for application to the skin or hair. Suitable surfactants for use herein include any known or otherwise effective cleansing surfactant suitable for application to the skin, and which is otherwise compatible with the other essential ingredients in the multi-phased personal care composition including water. These surfactants include anionic, nonionic, cationic, zwitterionic or amphoteric surfactants, soap or combinations thereof.

The multi-phased personal care composition preferably comprises a surfactant component at concentrations ranging from about 2% to about 99%, more preferably from about 10% to about 90%, even more preferably from about 25% to about 88%, still more preferably from about 40% to about 85%, and still even more preferably from about 45% to about 85%, by weight of the multi-phased personal care composition. The preferred pH range of the multi-phased personal care composition is from about 4 to about 9, more preferably about 6. The surfactant component in the present invention exhibits Non-Newtonian shear thinning behavior.

Non-limiting examples of surfactants for use in the compositions of the present invention are disclosed in McCutcheon's, Detergents and Emulsifiers, North American edition (1986), published by allured Publishing Corporation; and McCutcheon's, Functional Materials, North American Edition (1992); and in U.S. Pat. No. 3,929,678.

Anionic surfactants suitable for use in the cleansing phase include alkyl and alkyl ether sulfates. These materials have the respective formula ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohols having from about 8 to about 24 carbon atoms. Preferably, R has from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with about 1 to about 10, preferably from about 3 to about 5, and more preferably with about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the cleansing phase are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, preferably about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Preferred anionic surfactants for use in the cleansing phase include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants may be used in some embodiments.

Additional surfactant from the classes of amphoteric, zwitterionic surfactant, cationic surfactant, and/or nonionic surfactant may be incorporated in the cleansing phase compositions.

Amphoteric surfactants suitable for use in the cleansing phase include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecylaminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Zwitterionic surfactants suitable for use in the cleansing phase include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula: 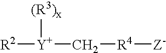
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the cleansing phase include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Amphoacetates and diamphoacetates may also be used. 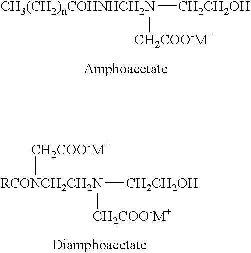
Amphoacetates and diamphoacetates conform to the formulas (above) where R is an aliphatic group of 8 to 18 carbon atoms. M is a cation such as sodium, potassium, ammonium, or substituted ammonium and n is an integer that varies from 8 to 20. Sodium lauroamphoacetate, sodium cocoamphoactetate, disodium lauroamphoacetate, and disodium cocodiamphoacetate are preferred in some embodiments.

Cationic surfactants can also be used in the cleansing phase, but are generally less preferred, and preferably represent less than about 5% by weight of the compositions.

Suitable nonionic surfactants for use in the aqueous cleansing phase include condensation products of alkylene oxide groups (hydrophilic in nature) with an organic hydrophobic compound, which may be aliphatic or alkyl aromatic in nature.

In an alternate embodiment of the present invention the cleansing phase comprises a surfactant component comprising a mixture of at least one nonionic surfactant, at least one anionic surfactant and at least one amphoteric surfactant, and an electrolyte.

Non-Ionic Surfactants

In an alternate embodiment of the present invention the multi-phased personal care composition can comprises at least one nonionic surfactant. Preferably the nonionic surfactant has an HLB from about 1.0 to about 20.0, more preferably from about 3.4 to about 15.0. The multi-phased personal care composition preferably comprises a nonionic surfactant at concentrations ranging from about 0.01% to about 50%, more preferably from about 0.10% to about 10%, and even more preferably from about 0.5% to about 5.0%, by weight of the surfactant component.

Nonionic surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, and mixtures thereof. In a preferred embodiment the nonionic surfactant is selected from the group consisting of glyceryl monohydroxystearate, Steareth-2, propylene glycol stearate, PEG-2 stearate, sorbitan monostearate, glyceryl stearate, laureth-2 and mixtures thereof. In a preferred embodiment the nonionic surfactant is Steareth-2.

Nonionic surfactants also useful herein include, lauramine oxide, cocoamine oxide.

Anionic Surfactants

In the alternate embodiment of the present invention the multi-phased personal care composition can comprises at least one anionic surfactant. Nonlimiting examples of suitable anionic surfactant were discussed previously.

Amphoteric Surfactants

In the alternate embodiment of the present invention the multi-phased personal care composition can comprises at least one amphoteric surfactant. Nonlimiting examples of suitable amphoteric surfactant were discussed previously.

Electrolyte

The electrolyte, if used, can be added per se to the multi-phased personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride or sodium or ammonium sulfate. A preferred electrolyte is sodium chloride. The electrolyte is preferably added to the surfactant component of the composition.

The electrolyte, when present, should be present in an amount, which facilitates formation of the stable composition (Non-Newtonian shear thinning behavior). Generally, this amount is from about 0.1% by weight to about 15% by weight, preferably from about 1% to about 6% by weight of the multi-phased personal care, but may be varied if required.

In another alternative embodiment of the present invention, the surfactant for use in the cleansing phase can be mixtures of surfactants. Suitable surfactant mixtures can comprise water, at least one anionic surfactant as described previously, an electrolyte as described previously, and at least one alkanolamide. The alkanolamide if present has the general structure of: 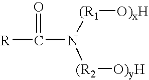
wherein R is C₈ to C₂₄, or preferably in some embodiments C₈ to C₂₂ or in other embodiments C₈ to C₁₈, saturated or unsaturated, straight chain or branched, aliphatic group; R₁ and R₂ are the same or different C₂-C₄ straight chain or branched aliphatic group; x is from 0 to 10; y is from 1 to 10; and wherein the sum of x and y is less than or equal to 10.

The amount of alkanolamide in the composition is typically about 0.1% to about 10%, by weight of the cleansing phase, and in some embodiments is preferably from about 2% to about 5%, by weight of the cleansing phase. Suitable alkanolamides include Cocamide MEA (Coco monethanolamide) and Cocamide MIPA (Coco monoisopropranolamide).

Non-Lathering Structured Aqueous Phase

The multi-phase personal care compositions of the present invention can comprise a non-lathering structured aqueous phase. The non-lathering structured aqueous phase of the composition comprises a water structurant and water. The non-lathering structured aqueous phase can be hydrophilic and in a preferred embodiment the non-lathering structured aqueous phase is a hydrophilic gelled water phase. In addition, the non-lathering structured aqueous phase typically comprises less than about 5%, preferably less than about 3%, and more preferably less than about 1%, by weight of the non-lathering structured aqueous phase, of a surfactant. In one embodiment of the present invention, the non-lathering structured aqueous phase is free of surfactant in the formulation.

The non-lathering structured aqueous phase of the present invention comprises from about 30% to about 99%, by weight of the non-lathering structured aqueous phase, of water. The non-lathering structured aqueous phase generally comprises more than about 50%, preferably more than about 60%, even more preferably more than about 70%, still more preferably more than about 80%, by weight of the non-lathering structured aqueous phase, of water.

The non-lathering structured aqueous phase will typically have a pH of from about 5 to about 9.5, more preferably about 7. The non-lathering structured aqueous phase can optionally comprise a pH regulator to facilitate the proper pH range.

A water structurant for the non-lathering structured aqueous phase can have a net cationic charge, net anionic charge, or neutral charge. In a preferred embodiment, the water structurant for the non-lathering structured aqueous phase has a net anionic charge.

The non-lathering structured aqueous phase of the present compositions can further comprise optional ingredients such as those described hereinafter. Preferred optional ingredients for the non-lathering structured aqueous phase include pigments, pH regulators, and preservatives. In one embodiment, the non-lathering structured aqueous phase comprises a water structurant (e.g. acrylates/vinyl isodecanoate crosspolymer), water, a pH regulator (e.g. triethanolamine), and a preservative (e.g. 1,3-dimethylol-5,5-dimethylhydantoin (“DMDMH” available from Lonza under the trade name GLYDANT®)).

A) Water Structurant

The non-lathering structured aqueous phase comprises from about 0.1% to about 30%, preferably from about 0.5% to about 20%, more preferably from about 0.5% to about 10%, and even more preferably from about 0.5% to about 5%, by weight of the non-lathering structured aqueous phase, of a water structurant.

The water structurant is typically selected from the group consisting of inorganic water structurants, charged polymeric water structurants, water soluble polymeric structurants, associative water structurants, and mixtures thereof.

Non-limiting examples of inorganic water structurants for use in the multi-phased personal care composition include silicas, clays such as synthetic silicates (Laponite XLG and Laponite XLS from Southern Clay), polymeric gellants such as polyacrylates, polyacrylamides, starches, modified starches, crosslinked polymeric gellants, copolymers, or mixtures thereof.

Non-limiting examples of charged polymeric water structurants for use in the multi-phased personal care composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), or mixtures thereof.

Non-limiting examples of water soluble polymeric structurants for use in the multi-phased personal care composition include cellulosic gel, hydroxypropyl starch phosphate (Structured XL from National Starch), polyvinyl alcohol, or mixtures thereof.

Non-limiting examples of associative water structurants for use in the multi-phased personal care composition include xanthum gum, gellum gum, pectin, alginate, or mixtures thereof.

Particle

The multi-phased personal care composition can comprise a particle. A water insoluble solid particle of various shapes and densities is useful. In a preferred embodiment, the particle tends to have a spherical, an oval, an irregular, or any other shape in which the ratio of the largest dimension to the smallest dimension (defined as the Aspect Ratio) is less than about 10. More preferably, the Aspect Ratio of the particle is less than about 8, still more preferably the Aspect Ratio of the particle is less than about 5.

The multi-phased personal care composition of the present invention can comprise the particle at a cosmetically efficacious level. Preferably, the particles are present from at least about 0.1% by weight of the composition, more preferably at least about 0.2% by weight of composition, even more preferably at least about 0.5%, still more preferably at least about 1%, and even still more preferably at least 2% by weight of the composition. In the multi-phased personal care composition of the present invention, preferably the particles comprises no more than about 50% by weight of composition, more preferably no more than about 30%, still more preferably no more than about 20%, and even more preferably no more than about 10% by weight of composition.

Preferably, the particle will also have physical properties which are not significantly affected by typical processing of the composition. Preferably, a particle having a melting point greater than about 70° C. is used, more preferably having a melting point greater than about 80° C., and even more preferably having a melting point of greater than about 95° C. is used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Non limiting examples of natural particles include various precipitated silica particles in hydrophilic and hydrophobic forms available from Degussa-Huls under the trade name Sipernet. Precipitated™, hydrophobic, synthetic amorphous silica, available from Degussa under the trade name Sipernet D11™ is a preferred particle. Snowtex colloidal silica particles available from Nissan Chemical America Corporation.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501 (nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

Exfoliant Particle

The exfoliant particle is selected from the group consisting of polyethylene, microcrystalline wax, jojoba esters, amorphous silica, talc, tracalcium orthophosphate, or blends thereof, and the like. The exfoliant particle has a particle size dimension along the major axis of the particle of from about 100 microns to about 600 microns, preferably from about 100 microns to about 300 microns. The exfoliant particle has a hardness of less than about 4 Mohs, preferably less than about 3 Mohs. The hardness as so measured is a criterion of the resistance of a particular material to crushing. It is known as being a fairly good indication of the abrasive character of a particulate ingredient. Examples of materials arranged in increasing order of hardness according to the Moh scale are as follows: h(hardness)-1: talc; h-2: gypsum, rock salt, crystalline salt in general, barytes, chalk, brimstone; h-4: fluorite, soft phosphate, magnesite, limestone; h-5: apatite, hard phosphate, hard limestone, chromite, bauxite; h-6: feldspar, ilmenite, hornblendes; h-7: quartz, granite; h-8: topaz; h-9: corrundum, emery; and h-10: diamond.

Preferably, the exfoliant particle has a color distinct from the cleansing base. The exfoliant particle is preferably present at a level of less than about 10%, preferably less than about 5%, by wt of the composition.

Shiny Particles

The multi-phased personal care composition can comprise a shiny particle. In a preferred embodiment the shiny particle is present in at least one phase of the multi-phase personal care composition. Nonlimiting examples of shiny particles include the following: interference pigment, multi-layered pigment, metallic particle, solid and liquid crystals, or combinations thereof.

An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer. The interference pigments of the multi-phased personal care compositions preferably comprises no more than about 20 weight percent of the composition, more preferably no more than about 10 weight percent, even more preferably no more than about 7 weight percent, and still more preferably no more than about 5 weight percent of the multi-phased personal care composition. The interference pigment of the multi-phased personal care composition preferably comprises at least about 0.1 weight percent of the multi-phased personal care composition, more preferably at least about 0.2 weight percent, even more preferably at least about 0.5 weight percent, and still more preferably at least about 1 weight percent by weight of the multi-phased personal composition.

The interference pigment can comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃SnO₂. For the multiple layer structures, the thin films can be consisted of all high refractive index materials or alternation of thin films with high and low RI materials with the high RI film as the top layer.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine. It was found that the color of the interference pigment in addition to its brightness had a significant influence on human perception of skin tone. In general, preferred colors are silver, gold, red, green and mixtures thereof.

Non-limiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The hydrophobically modified interference pigment or HMIP allows for the entrapment of the HMIP within the phases and greater deposition of the HMIP. Preferably the ratio of HMIP to a phase is 1:1 to about 1:70, more preferably 1:2 to about 1:50, still more preferably 1:3 to about 1:40 and most preferably 1:7 to about 1:35.

In an embodiment of the present invention the HMIP's are preferably entrapped within the hydrophobic composition. This necessitates that the hydrophobic composition particle size is generally larger than the HMIP. In a preferred embodiment of the invention, the hydrophobic composition particles contain only a small number of HMIPs per hydrophobic composition particles. Preferably this is less than 20, more preferably less than 10, most preferably less than 5. These parameters, the relative size of the benefit droplets to the HMIP and the approximate number of HMIP particles per hydrophobic composition particles, can be determined by using visual inspection with light microscopy.

The HMIP of the present invention preferably has a hydrophobic coating comprising at least about 0.1 weight percent of the total particle weight, more preferably at least about 0.5 weight percent, even more preferably at least about 1 weight percent. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

Optional Ingredients

A variety of suitable optional ingredients can be employed in the multi-phase personal care composition. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional materials can be used in any aspect of the compositions of the present invention, including each phase as described herein.

Non-limiting optional ingredients include humectants and solutes. A variety of humectants and solutes can be employed and can be present at a level of from about 0.1% to about 50%, preferably from about 0.5% to about 35%, and more preferably from about 2% to about 20%, by weight of the personal care composition. A preferred humectant is glycerin.

A preferred water soluble, organic material is selected from the group consisting of a polyol of the structure:
R1-O(CH₂—CR2HO)ₙH
where R1=H, C1-C4 alkyl; R2=H, CH₃ and n=1-200; C2-C10 alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and ethanol amines of the general structure (HOCH₂CH₂)ₓNH_{y} where x=1-3; y=0-2, and x+y=3, and mixtures thereof. The most preferred polyols are selected from the group consisting of glycerine, polyoxypropylene(1) glycerol and polyoxypropylene(3) glycerol, sorbitol, butylene glycol, propylene glycol, sucrose, urea and triethanol amine.

Nonionic polyethylene/polypropylene glycol polymers are preferably used as skin conditioning agents. Polymers useful herein that are especially preferred are PEG-2M wherein x equals 2 and n has an average value of about 2,000 (PEG 2-M is also known as Polyox WSR® N-10 from Union Carbide and as PEG-2,000); PEG-5M wherein x equals 2 and n has an average value of about 5,000 (PEG 5-M is also known as Polyox WSR® 35 and Polyox WSR® N-80, both from Union Carbide and as PEG-5,000 and Polyethylene Glycol 200,000); PEG-7M wherein x equals 2 and n has an average value of about 7,000 (PEG 7-M is also known as Polyox WSR® (N-750 from Union Carbide); PEG-9M wherein x equals 2 and n has an average value of about 9,000 (PEG 9-M is also known as Polyox WSR® N-3333 from Union Carbide); PEG-14 M wherein x equals 2 and n has an average value of about 14,000 (PEG 14-M is also known as Polyox WSR®205 and Polyox WSR® N-3000 both from Union Carbide); and PEG-90M wherein x equals 2 and n has an average value of about 90,000. (PEG-90M is also known as Polyox WSR®-301 from Union Carbide.)

Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Flow Onset Temperature Method

The Flow Onset Temperature for a benefit phase can be characterized by applying a steady stress during a temperature increase using a rheometer such as a TA Instruments AR2000. The Flow Onset Temperature is determined in the following manner.

First, the benefit phase is obtained in sufficient quantity to measure on a rheometer with 40 mm diameter plate and 1 mm gap (e.g., about several grams). When the benefit phase is a part of an inhomogeneous composition, and can be separated from the other components of the composition by physical forces, such as centrifugation, without significantly altering the composition (see, e.g., Examples herein), the benefit phase is separated. If the benefit phase is physically different in the composition than when prepared separately, such as might result from processing, then the benefit phase from the composition is evaluated. If more than one distinct (immiscible, e.g.) benefit phases exists, such as for example a silicone oil benefit phase and a hydrocarbon benefit phase, they are evaluated separately from each other, even if one is an emulsion within the other, unless the phases are inseparable by physical forces without substantially altering the balance of the benefit phase and the individual phases cannot be reconstituted separately.

For measurement, a parallel plate geometry is used with a 1 mm gap setting at a starting temperature of 25° C. A sample of the benefit phase is loaded onto the rheometer base plate using a spatula to minimize shear, and the upper plate is lowered into position 1 mm above the base plate. The upper plate is locked so as not to rotate, and a square edged spatula is used to remove excess composition at the edge of the plate, then unlocked. The sample is allowed to equilibrate for 2 minutes. Next, the rheometer applies a steady rotational stress of 0.25 Pascals (Pa) while, at the same time, temperature is increased from 25° C. to 70° C. at a rate of 4.0° C./minute. Viscosity data are obtained in 1:5 Pascal-seconds (Pa-s), approximately one data point every 5 seconds during the measurement. After completion of the measurement, a first median viscosity is obtained between 25 and 38° C. by taking the median of the viscosity data between those temperatures. The data points are then examined and all viscosity data points having a viscosity value greater than five times the first median viscosity are removed from the data. Subsequently, a second median viscosity is obtained by taking the median of the remaining viscosity data points between 25 and 38° C. The viscosity data are examined to determine the temperature at which the viscosity falls below one quarter of the second median viscosity. If the viscosity falls below ¼ of the second median more than once, the last time it falls is used. If the viscosity never falls below ¼ of the second median viscosity, the point at which it falls below half the second median is used instead. Starting with the data point at this temperature, a regression is performed over the following 10° C. interval of the data. First, the log₁₀ of the Temperature (° C.) is obtained for the data points in this temperature interval; then the log₁₀ of the corresponding viscosity (Pa-s) data are obtained. A linear least squares regression of the general form y=m×+b is performed on the logarithmically transformed data, obtaining an equation of the form log(Temperature)=m*log(viscosity)+b. The Flow Onset Temperature is obtained by determining the intersection of the regression obtained with the second median viscosity, by substituting the second median viscosity into the regression equation for ‘viscosity’ and solving for ‘Temperature’, then converting the result obtained to degrees Fahrenheit. The Flow Onset Temperature reported is an indication of a temperature below which the composition should remain in order to be most stable during shipping and handling.

Structural Rigidity Method

To measure rigidity of a benefit phase, a stress can be applied rotationally to the benefit phase using a rheometer, and the relationship between stress and strain at a low value of stress, 1 Pa, expressed as the stress divided by the resulting amount of strain the benefit phase experiences. The Structural Rigidity is defined as 1 Pa divided by the amount of unitless strain demonstrated at 1 Pa of stress in this test, at 100° F.

First, the benefit phase is obtained in sufficient quantity to measure on a rheometer with 40 mm diameter plate and 1 mm gap (e.g., about several grams). When the benefit phase is a part of an inhomogeneous composition, and can be separated from the other components of the composition by physical forces, such as centrifugation, without significantly altering the composition (see, e.g., Examples herein), the benefit phase is separated. If the benefit phase is physically different in the composition than when prepared separately, such as might result from processing, then the benefit phase from the composition is evaluated. If more than one distinct (immiscible, e.g.) benefit phases exists, such as for example a silicone oil benefit phase and a hydrocarbon benefit phase, they are evaluated separately from each other, even if one is an emulsion within the other.

To measure Structure Rigidity, a controlled stress rheometer such as a TA Instruments AR2000 rheometer (TA Instruments, 109 Lukens Drive, New Castle, Del., USA) with a 40 mm steel plate at a gap of 1 mm is used with a Peltier heating mechanism in the base plate, and a lower plate alloy designed for high heat transfer to the sample. The rheometer is stationed in a room with still air at an air temperature of not less than 75° F. in order to minimize heat losses at the sample top. A sample of the benefit phase is loaded onto the rheometer base plate using a spatula to minimize shear, and the upper plate is lowered into position 1 mm above the base plate. The upper plate is locked so as not to rotate, and a square edged spatula is used to remove excess composition at the edge of the plate, then unlocked. The temperature is raised to 100° F. using the rheometer software within 30 seconds of loading, and once so reached the sample is allowed to equilibrate for 3 minutes. The rheometer then starts to rotate at a rotational stress of 0.1 Pa and increases stress to 100 Pa over a 3 minute interval in a logarithmic progression (i.e., stress is increased by 3 decades which is 3 log units in this test, over 3 minutes, hence the rate of increase is 1 log unit of stress per minute), collecting a data point 15 times per decade of stress, or 45 total points. The strain, initially at zero to start the experiment, is measured. The strain at 1 Pa of stress is determined from the results by interpolating (linearly) the strain at 1 Pa from the points on either side of 1 Pa when a point does not coincide precisely with 1 Pa. Strain and stress are indicated at the sample edge, as is conventional with parallel plate geometries. Structure Rigidity, in Pa, is determined by dividing 1 Pa of stress by the strain at 1 Pa according to the following equation.
Structure Rigidity=(1 Pa)/(strain at 1 Pa)
Method of Use

The multi-phase personal care compositions of the present invention are preferably applied topically to the desired area of the skin or hair in an amount sufficient to provide effective delivery of the skin cleansing agent, hydrophobic component, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water.

The present invention is therefore also directed to methods of cleansing the skin through the above-described application of the compositions of the present invention. The methods of the present invention are also directed to a method of providing effective delivery of the desired skin active agent, and the resulting benefits from such effective delivery as described herein, to the applied surface through the above-described application of the compositions of the present invention. Preferably, the method is directed to delivering skin benefits to skin or hair, said method comprising the steps of: dispensing an effective amount of a multi-phase personal care composition according to the multi-phased personal care composition of the present invention onto an implement selected from the group consisting of a cleansing puff, washcloth, sponge, and human hand; topically applying said composition to said skin or hair using said implement; and removing said composition from said skin or hair by rinsing said skin or hair

Method of Manufacture

The multi-phase personal care compositions may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples.

If the multi-phase personal care compositions comprises patterns of varying colors it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

Article of Commerce

While not wishing to be bound by theory, it is believed that efficacy of the product can be linked to the ability of the consumer to understand the usage instructions and to use the product accordingly. The instruction set included may contain pictures or illustrations of the product being applied as well as written instructions. Therefore, the present invention also relates to an article of commerce comprising (a) a container having an s-wave orifice; (b) a multi-phase personal care composition contained in said container having an s-wave orifice; and (c) a set of instructions in association with said container having an s-wave orifice; and said set of instructions comprising instructions to cleanse, to condition, to moisturize the skin and rinse as normal; and wherein said container having an s-wave orifice allows for easy dispensing of said composition.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

A petrolatum can be obtained from Witco division of Crompton Corporation (Petrolia, Pa., USA) which is a bleached USP petrolatum, and is sold under the trade name Super White Protopet. The petrolatum has a complete melting point of 137° F. by ASTM D127, a specific gravity of 0.84, a Saybold viscosity at 99° C. of 74.0 cP. A gas chromatogram (GC) of the petrolatum indicates hydrocarbons between C12 and C120 are present. Taking the ratio of the average peak heights of the GC for hydrocarbons having even numbered chain lengths from C22-28, C44-50 and C94-116, the petrolatum has a ratio of peak heights of 1.5:1.0:0.25. The petrolatum can be milled with 0.05% of FD&C Red No. Calcium 7 Lake to make a benefit phase, disperse the pigment at a high shear rate in the petrolatum above its melting point, then cool to ambient temperature in a jar. The benefit phase, together with a cleansing phase, can be prepared as a personal care composition. The cleansing phase can be prepared from the following ingredients:

### Cleansing Phase

| Ingredients | Ingredient wt % |
|---|---|
| Sodium Trideceth Sulfate (as Rhodapex EST-30, | 14.8 |
| 30% active, Rhodia) | |
| Sodium Lauroamphoacetate (Miranol Ultra L-32, | 5.9 |
| 32% active, Rhodia) | |
| Cocamide MEA (Alkamide C-212, Rhodia) | 3.0 |
| Guar Hydroxypropyltrimonium Chloride | 0.7 |
| (N-Hance 3196 from Aqualon) | |
| PEG 90M (Polyox WSR 301 from Dow Chemical) | 0.1 |
| Glycerin | 0.3 |
| Sodium Chloride | 3.5 |
| Disodium EDTA | 0.05 |
| Glydant (DMDMH, preservative, Lonza Inc.) | 0.67 |
| Citric Acid | 0.4 |
| Perfume | 2.0 |
| Expancel 091 DE 40 d30 (from Expancel, Inc.) | 0.45 |
| gas filled spheres, 50 micron | |
| Water | Q.S. to 100% |

Prepare the cleansing phase; first, add citric acid into water at 1:3 ratio to form a citric acid premix. Disperse Polyox WSR 301 in glycerin at 1:3 ratio to form a Polyox premix. Disperse Polymer N-Hance in water at 1:10 ratio to form a N-Hance premix. Then, add the following ingredients into the main mixing vessel in the following sequence: water, N-Hance premix, Polyox premix, citric acid premix, and Expancel. Start agitation of the mixing vessel. Then, add surfactants, sodium benzoate, disodium EDTA, glydant, and perfume and blend. Add sodium chloride and mix until well dispersed. Keep agitating until a homogenous solution forms which has a gel like character and is able to suspend large air bubbles (5 mm, e.g.). Adjust pH to 6.0.

The cleansing phase and the benefit phase can be blended to make a multi-phased personal care composition with a striped pattern. Heat the benefit phase to 80° C., pump through a plate and frame heat exchanger to obtain an outlet temperature of 43° C., then pipe through a side injection port of a ¾ inch diameter pipe just upstream from a Koch-Glitsch (Koch-Glitsch Co., Wichita, Kans., USA) 4 element static mixer. Pump the cleansing phase at 27° C. through the piping and static mixer, the static mixer serving to meld the phases without homogenizing or creating a dominant continuous phase of either. The mass flow rate of the two streams is comparable. At the static mixer outlet, fill a 13.5 oz. oblate cylindrical flat bottomed PET bottle with composition starting at the bottom in about 3 seconds while spinning the bottle at a rate of about 250 rpm to create a generally horizontal striped pattern visible from the package exterior through the bottle, which is transparent PET. The bottle is about 190 mm from bottom to shoulder and about 70 mm wide at the widest point midway from base to neck, and capped with a cap having a 7 mm diameter round centrally located orifice. The empty bottle weighs about 34 grams without cap. Wash the skin with the composition and dry the skin off by patting dry with a towel, wait 10 minutes, the skin has a noticeably more draggy feel on the surface compared to the same unwashed skin. Dispense fifty grams of the composition into a 4-ounce cylindrical glass jar having inner diameter of about 45 mm and centrifuged at 3000 rpm for 22 minutes using a Heraeus Instruments Labofuge 400 centrifuge (10 cm arm radius measured to sling axis, Heraeus Instruments, 1-800-437-2387, USA) to separate the phases into distinct layers while not separating the components within a phase (e.g., crystals of wax from mineral oil within the benefit phase), with the exception of air or air-containing components (gas filled spheres, e.g.). If there is evidence, visual or otherwise (e.g., the sample measures much differently in different locations), of segregation of components within the benefit phase, the separation procedure is repeated with a new 40 gram sample, reducing the rpm by 500 and/or the time by 4 min progressively until a separated sample can be obtained that does not exhibit segregation within the benefit phase. Expancel spheres, having a very low density, float to the top and are discarded. Remove the benefit phase from the jar without shearing excessively using an L-shaped spatula, and Consistency Value is measured. The benefit phase has an average Consistency Value of about 305 Pa-sec, a shear index of about 0.24, a Flow Onset Temperature of 100° F. and a Structure Rigidity of 190 Pascals (Pa).

This composition has very good stability at high temperatures.

### Example 2

Blend mineral oil with the benefit phase of Example 1 to improve the skin feel and dispensing. Obtain the petrolatum of Example 1. Obtain mineral oil having a Consistency Value of about 0.7 Pa-sec and a shear index of about 0.98 from Witco, which is sold as Hydrobrite 1000. The mineral oil has a carbon number profile with essentially no chains under 29 carbon units long. By weight, 60% Super White Protopet is mixed with 40% Hydrobrite 1000 mineral oil at 90° C. and the resulting mixture processed with pigment in the same manner as Example 1 to make a benefit phase. A multi-phased personal care composition can be prepared in the same manner as Example 1. Obtain a separation of the benefit from cleansing phase using the centrifuge at 3000 rpm for 22 minutes. The benefit phase is measured and has a Consistency Value of 78 Pa-sec, a shear index of 0.35, a Flow Onset Temperature of 99.6° F. and a Structure Rigidity of 60 Pa.

This composition has good stability at high temperatures. After use, it leaves the skin surface feeling moisturized but not draggy.

### Example 3

A multi-phased personal care composition can be prepared using the benefit phase of Example 2 with a weight ratio of cleansing phase:benefit phase of 80:20. After tempering, a separation is accomplished in the centrifuge at 3000 rpm for 12 minutes. Two jars are prepared since there is less benefit phase in this example than in Example 2. The benefit phase has a Consistency Value of 57 Pa-sec, and a shear index of 0.41. After use, the multi-phased personal care composition leaves the skin surface feeling moisturized but not draggy. The composition is easy to dispense, has good stability, and a moisturized feel on the skin after use which is preferred.

### Examples 4-7

The benefit phase of Example 2 can be prepared four separate times as four separate benefit phases. Multi-phased personal care compositions can be prepared using each benefit phase with the cleansing phase composition of Example 1 by blending at a 50:50 ratio according to the procedure of Example 1, varying the total flow rate according to the following table (fill time is allowed to vary with flow rate). After tempering, the compositions can be separated using the centrifuge with resulting benefit phase. Consistency Value can be measured for the benefit phase. The Consistency Value can be measured for Example 4 in several locations within the separated benefit phase and is found to vary slightly from the top of the separated benefit phase to the bottom of the separated benefit phase. E.g., in one test 97.2 Pa-sec is obtained from the top portion of the benefit phase and 77.1 Pa-sec from the lower portion of the benefit phase. This amount of variation, even if systemic, can be considered normal since there is no visible evidence of striation within the phase and the difference from the mean is less than about 20% of the mean for all samples, so adjusting centrifuge conditions and repeating the measurements is not necessary, and average values are reported by sampling the same number of samples at the top, middle and bottom of the benefit phase to obtain a representative mean value.

| | | | Number of | Consistency | | Average | |
|---|---|---|---|---|---|---|---|
| | Total Flow Rate | | bottles | Values obtained | Shear Indices | Consistency | Average |
| Example | of both phases | | tested | (Pa-sec) | obtained | Value (Pa-sec) | Shear Index |
| 4 | 3 | kg/min | 3 (2 | 97.2, 77.1, | 0.33, 0.36, | 84.1 | 0.35 |
| | | | tests per | 91.8, 74.6, | 0.34, 0.35, | | |
| | | | bottle) | 81.2, 82.4 | 0.37, 0.37 | | |
| 5 | 3 | kg/min | 1 | 70.4 | 0.39 | 70.4 | 0.39 |
| 6 | 4.5 | kg/min | 1 | 50.0, 67.6 | 0.39, 0.43 | 58.8 | 0.41 |
| 7 | 4.5 | kg/min | 1 | 66.7 | 0.41 | 66.7 | 0.41 |

Example 4 has a Flow Onset Temperature of 102.4° F. and a Structure Rigidity of 79 Pa. Example 6 has a Flow Onset Temperature of 103° F. and a Structure Rigidity of 59.5 Pa. These examples have good stability at high temperatures and after use leave the skin surface feeling moisturized but not draggy or sticky.

### Example 8

The benefit phase of Example 2 can be prepared, then heat to 90° C. and add 2.5% by weight of microcrystalline wax which is sold by Witco under the trade name W445 microcrystalline wax and which has a melting point of 178° F. (ASTM D127 melt point method) a penetration hardness of 27 (dmm, ASTM D1321 method) and a Saybold viscosity at 210° F. of 86 (SUS, ASTM D2161 method) to 97.5% of the benefit phase, and mix until melted. Pump the phase through a plate and frame heat exchanger to obtain an outlet temperature of 43° C. and a multi-phased personal care composition can be prepared according to the procedure of Example 1 using the cleansing phase, which is maintained at a temperature of 15° C. prior to combining. After tempering, the composition is separated (3000 rpm, 21 minutes). The benefit phase has an average Consistency Value of 84.4 Pa-sec and a shear index of 0.36, a Flow Onset Temperature of 101.9° F. and a Structural Rigidity of 229 Pa.

### Example 9

The composition and procedure of Example 8 can be followed except that the cleansing phase is held at 27° C. prior to combining and the heat exchanger outlet temperature of the benefit phase is 49° C. The benefit phase has an average Consistency Value of 74.1 Pa-sec, a shear index of 0.39, a Structure Rigidity of 204 Pa, and a Flow Onset Temperature of 102.3° F.

### Example 10

The composition and procedure of Example 8 can be followed except that the cleansing phase is held at 36° C. prior to combining and the heat exchanger outlet is 46° C. The resulting composition pattern is marbled due to the small width of the stripes and more random, less horizontal, stripe direction. The benefit phase has a Consistency Value of 87.8 Pa-sec and a shear index of 0.37, a Structure Rigidity of 264 Pa, and a Flow Onset Temperature of 104.8° F.

### Example 11

A benefit phase can be prepared using 97.2% Super White Protopet petrolatum, 2.0% W445 microcrystalline wax and 0.8% paraffin wax 1246, all from Crompton-Witco, by milling with pigment in the manner described in Example 1. A multi-phased personal care composition can be prepared using 50% of the cleansing phase of Example 1 with 50% benefit phase in the manner described in Example 1. Temper and separate the phases, then, the benefit phase is measured and has an average Consistency Value of 130 Pa-sec with a coefficient of variation of 11% and a shear index of 0.30, a Structure Rigidity of 186 Pa, and a Flow Onset Temperature of 114.2° F. After using the multi-phased personal care composition, the skin has a draggy feel.

### Examples 12

A benefit phase can be prepared in the manner of Example 2 substituting Hydrobrite 550 mineral oil (Crompton Witco) in place of Hydrobrite 1000. Hydrobrite 550 is lower in viscosity than Hydrobrite 1000, and has essentially no hydrocarbons below a chain length of 27 carbons as determined by GC. A multi-phased personal care composition can be prepared using 50-50 weight ratio of benefit phase to cleansing phase using the cleansing phase of Example 1 in the manner described in Example 2. Temper and separate, then, the benefit phase is measured and has a Consistency Value of 38 Pa-sec with a shear index of 0.43, a Structure Rigidity of 59 Pa, and a Flow Onset Temperature of 94.2° F. The composition has a preferred skin feel after use. The composition has moderate thermal stability.

### Example 13

A benefit phase can be prepared by adding 6% W445 microcrystalline wax into 94% of the petrolatum-mineral oil mixture of Example 12, melt the wax as previously described and then cool and temper. Temper and separate, then, the benefit phase is measured and has a Consistency Value of 75 Pa-sec with a shear index of 0.365, a Structure Rigidity of 225 Pa, and a Flow Onset Temperature of 104.8° F. The composition has a good feel on the skin after use, and has very good thermal stability.

### Example 14

A petrolatum can be obtained from Witco division of Crompton Corporation (Petrolia, Pa., USA) which is a bleached petrolatum sold under the name G2218 Petrolatum. The petrolatum has a complete melting point of about 139° F., a Saybold viscosity of between about 75-86 SUS at 210° F., a Penetration of between 192-205 dmm, a Consistency Value of about 42 Pa-s with a shear index of about 0.53, a Structure Rigidity of 370 Pa and a Flow Onset Temperature of 109.8° F. A gas chromatogram of the petrolatum indicates hydrocarbons between C20 and C120 are present. Taking the ratio of the average peak heights of the GC for hydrocarbons having even numbered chain lengths from C22-28, C44-50 and C94-116, the petrolatum has a ratio of peak heights of about 0.72:1.0:0.32. Blend the petrolatum at 80° C. with Hydrobrite 1000 mineral oil in a weight ratio of 70 parts petrolatum:30 parts mineral oil. Mill the hot mixture with 0.05% of FD&C Red No. Calcium 7 Lake to make a benefit phase. Pump the benefit phase through a heat exchanger with an outlet temperature of 43° C. and combine with equal weight cleansing phase of Example 1 in the same manner described in Example 1 to make a multi-phase personal care composition with a striped pattern. Temper and separate, then, the benefit phase is measured and has a Consistency Value of 54.1 Pa-s, a shear index of 0.40, a Structure Rigidity of 101 Pa, and a Flow Onset Temperature of 103° F. The benefit phase has a preferred moisturizing skin feel after use. The composition is stable at elevated temperatures.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase personal care composition comprising: at least two visually distinct phases;
wherein at least one visually distinct phase comprises a benefit phase; and
wherein said benefit phase has a Flow Onset Temperature of at least about 97° F.; and
wherein said benefit phase has a Consistency Value (K) from about 30 to about 350 Pa-s; and
wherein said visually distinct phases form a pattern; and
wherein said visually distinct phases are packaged in physical contact with one another; and
wherein said visually distinct phases are stable.

2. The multi-phase personal care composition of claim 1, wherein said benefit phase comprises a hydrophobic composition comprising a hydrophobic component.

3. The multi-phase personal care composition of claim 2, wherein said benefit phase comprises from about 5% to about 100%, by weight of said benefit phase, of said hydrophobic composition.

4. The multi-phase personal care composition of claim 2, wherein said hydrophobic composition comprises from about 5% to about 100%, by weight of said benefit phase, of said hydrophobic component.

5. The multi-phase personal care composition of claim 2, wherein said hydrophobic component is selected from the group consisting of natural and synthetic lipids, hydrocarbons, fats, oils, hydrophobic plant extracts, fatty acids, essential oils, silicone oils, triglycerides, and mixtures thereof.

6. The multi-phase personal care composition of claim 5, wherein said hydrophobic component is selected from the group consisting of petrolatum, mineral oil, microcrystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

7. The multi-phase personal care composition of claim 1, wherein said two visually distinct phases are selected from the group consisting of a cleansing phase, a benefit phase, a non-lathering structured aqueous phase and combinations thereof.

8. The multi-phase personal care composition of claim 7, wherein the cleansing phase comprises:
(i) at least one anionic surfactant;
(ii) at least one electrolyte;
(iii) at least one alkanolamide; and
(iv) water;
wherein the cleansing phase is non-Newtonian shear thinning; and
the cleansing phase has a viscosity of equal to or greater than about 3000 cps.

9. The multi-phase personal care composition of claim 1, wherein said pattern is selected from the group consisting of striped, geometric, marbled and combinations thereof.

10. The multi-phase personal care composition of claim 1, wherein said composition additionally comprises skin care actives selected from the group consisting of vitamins and derivatives thereof; sunscreens; preservatives; anti-acne medicaments; antioxidants; skin soothing and healing; chelators and sequestrants; essential oils, skin sensates, and mixtures thereof.

11. The multi-phase personal care composition of claim 1, wherein at least one phase comprises a particle

12. A multi-phase personal care composition comprising: at least two visually distinct phases;
wherein at least one visually distinct phase comprises a benefit phase; and
wherein said benefit phase has a Structural Rigidity of greater than about 60 Pa; and
wherein said benefit phase has a Consistency Value (K) from about 30 to about 350 Pa-s; and
wherein said phases form a pattern; and
wherein said phases are packaged in physical contact with one another; and
wherein said visually distinct phases are stable.

13. The multi-phase personal care composition of claim 12, wherein said benefit phase has a Structural Rigidity of at least about 65 Pa.

14. The multi-phase personal care composition of claim 12, wherein said benefit phase comprises a hydrophobic composition comprising from about 5% to about 100%, by weight of said benefit phase, of a hydrophobic component.

15. The multi-phase personal care composition of claim 14, wherein said hydrophobic component is selected from the group consisting of natural and synthetic lipids, hydrocarbons, fats, oils, hydrophobic plant extracts, fatty acids, essential oils, silicone oils, triglycerides, and mixtures thereof.

16. The multi-phase personal care composition of claim 15, wherein said hydrophobic component is selected from the group consisting of petrolatum, mineral oil, microcrystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

17. The multi-phase personal care composition of claim 12, wherein said two visually distinct phases are selected from the group consisting of a cleansing phase, a benefit phase, a non-lathering structured aqueous phase and combinations thereof.

18. The multi-phase personal care composition of claim 17, wherein the cleansing phase comprises:
i) at least one anionic surfactant;
(ii) at least one electrolyte;
(iii) at least one alkanolamide; and
(iv) water;
wherein the cleansing phase is non-Newtonian shear thinning; and
the cleansing phase has a viscosity of equal to or greater than about 3000 cps.

19. The multi-phase personal care composition of claim 12, wherein said composition additionally comprises skin care actives selected from the group consisting of vitamins and derivatives thereof; sunscreens; preservatives; anti-acne medicaments; antioxidants; skin soothing and healing; chelators and sequestrants; essential oils, skin sensates, and mixtures thereof.

20. The multi-phase personal care composition of claim 12, wherein at least one phase comprises a particle.

21. A multi-phase personal care composition comprising: at least two visually distinct phases comprising;
a) a benefit phase;
b) a cleansing phase comprising;
i) a surfactant component comprising;
a) at least one nonionic surfactant having an HLB from about 1.0 to about 20.0;
b) at least one anionic surfactant;
c) at least one amphoteric surfactant;
ii) an electrolyte; and
wherein said benefit phase has a Flow Onset Temperature of at least about 97° F.; and
wherein said benefit phase has a Consistency Value (K) from about 30 to about 350 Pa-s; and
wherein said phases form a pattern; and
wherein said phases are packaged in physical contact with one another; and
wherein said visually distinct phases are stable.

22. The multi-phase personal care composition of claim 21, wherein said two visually distinct phases are selected from the group consisting of a cleansing phase, a benefit phase, a non-lathering structured aqueous phase and combinations thereof.

23. The multi-phase personal care composition of claim 21, wherein said composition additionally comprises skin care actives selected from the group consisting of vitamins and derivatives thereof; sunscreens; preservatives; anti-acne medicaments; antioxidants; skin soothing and healing; chelators and sequestrants; essential oils, skin sensates, and mixtures thereof.

24. The multi-phase personal care composition of claim 21, wherein at least one phase comprises a particle

25. A method of delivering skin benefits to skin or hair, said method comprising the steps of:
a) dispensing an effective amount of a multi-phase personal care composition according to claim 1 onto an implement selected from the group consisting of a cleansing puff, washcloth, sponge, and human hand;
b) topically applying said composition to said skin or hair using said implement; and
c) removing said composition from said skin or hair by rinsing said skin or hair with water.

26. A method of delivering skin benefits to skin or hair, said method comprising the steps of:
a) dispensing an effective amount of a multi-phase personal care composition according to claim 21 onto an implement selected from the group consisting of a cleansing puff, washcloth, sponge, and human hand;
b) topically applying said composition to said skin or hair using said implement; and
c) removing said composition from said skin or hair by rinsing said skin or hair with water.

27. An article of commerce comprising
(a) a container having an s-wave orifice;
(b) a multi-phase personal care composition contained in said container having an s-wave orifice; and
(c) a set of instructions in association with said container having an s-wave orifice; and said set of instructions comprising instructions to cleanse, to condition, to moisturize the skin and rinse as normal; and
wherein said container allows for easy dispensing of said composition.
